(19) United States
(12) Patent Application Publication
(10) Pub. No.: US 2023/0181460 A1
(43) Pub. Date: Jun. 15, 2023

(54) PRESERVATIVE FREE PHARMACEUTICAL COMPOSITION FOR OPHTHALMIC ADMINISTRATION CONTAINING CYCLOSPORINE

(71) Applicant: PHARMATHEN S.A., Pallini Attikis (GR)

(72) Inventor: Evangelos KARAVAS, Attikis (GR)

(21) Appl. No.: 17/924,651

(22) PCT Filed: May 10, 2021
(86) PCT No.: PCT/EP2021/025177

§ 371 Date: Nov. 10, 2022

(30) Foreign Application Priority Data
May 12, 2020 (GR) 20200100244

(51) Int. Cl. A61K 9/00; A61K 38/13; A61K 9/107; A61K 47/44; A61K 47/26; A61K 47/32; A61K 47/10

(52) U.S. Cl. CPC A61K 9/0048; A61K 9/107; A61K 38/13; A61K 47/10; A61K 47/26; A61K 47/32; A61K 47/44

(57) ABSTRACT

The present invention relates to a stable preservative-free Cyclosporine emulsion in the form of eye drops and a process for the manufacturing thereof, packed in a container that ensures stability of the product for the treatment of keratoconjunctivitis sicca.

### TECHNICAL FIELD OF THE INVENTION

The present invention relates to a preservative free ophthalmic formulation for topical administration containing a therapeutically effective quantity of an immunosuppressive agent such as Cyclosporine to be used for the treatment of keratoconjunctivitis sicca (dry eyes) and the process for its preparation. Such preservative-free formulation is packed in container that ensures physical and chemical stability of the product.

### BACKGROUND OF THE INVENTION

Keratoconjunctivitis sicca (KCS) also known as Dry eye syndrome (DES) is one of the most common problems affecting the general population and can cause problems that range in severity from mildly irritating to debilitating.

Dry eye syndrome is a general term that describes the state of the front of the eye in response to a breakdown in the natural layer of tears that coats the front of the eye, called the tear film. Normally, this layer of tears is a stable, homogenous layer that not only provides the cornea and conjunctiva a healthy buffer from damage were it constantly exposed to the air, but this interface between the tear film and the air is also responsible for a significant amount of the focusing power of the eye. When the tear film becomes unhealthy it breaks down in different places on the cornea and conjunctiva, leading not only to symptoms of irritation, but also to unstable and intermittently changing vision. Other associated symptoms include redness, discharge, and easily fatigued eyes. Blurred vision may also occur. Scarring of the cornea may occur in some cases without treatment.

Inflammation occurring in response to tear film hypertonicity can be suppressed with topical immunosuppressants such as Cyclosporine.

Cyclosporine is an immunosuppressive agent when administered systemically. In patients whose tear production is presumed to be suppressed due to ocular inflammation associated with keratoconjunctivitis sicca, Cyclosporine emulsion is thought to act as a partial immunomodulator. The exact mechanism of action is not known.

The chemical name of Cyclosporine is (3S,6S,9S,12R,15S,18S,21S,24S,30S,33S)-30-Ethyl-33-[(lR,2R,4E)-l-hydroxy-2-methyl-4-hexen-l-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone. It is an off-white crystalline solid. Its molecular formula is C₆₂H₁₁₁N₁₁O₁₂ corresponding to a molecular weight of 1202.61. It is slightly soluble in water and saturated hydrocarbons; very soluble in methanol, acetone, and diethyl ether.

EP-B-1142566 discloses topical ophthalmological formulations comprising aqueous solutions containing Cyclosporine, hyaluronic acid or its salt and polysorbate 80.

US-B-6555526 discloses an ophthalmic pharmaceutical composition comprising trehalose as an effective ingredient and a pharmaceutically-acceptable carrier.

There still remains a need for an effective and safe topical ophthalmic pharmaceutical composition containing Cyclosporine with increased stability, improved solubility and fewer side effects. In particular, there is a need for an ophthalmic composition that is free from preservatives to be provided in a multiple use container and provide efficient dosing of the solution to the patient, without wastage.

### SUMMARY OF THE INVENTION

The main objective of the present invention is to develop a stable, preservative-free ophthalmic formulation comprising Cyclosporine to be used for the treatment of keratoconjunctivitis sicca providing a significant improvement over the prior art formulations.

Moreover, an aspect of the present invention is to provide a preservative free ophthalmic formulation for topical administration containing Cyclosporine which is bioavailable and effective with sufficient self-life.

A further approach of the present invention is to provide ophthalmic solutions that are easily administrable in drop form.

Furthermore, it is an object of the present invention to provide an ophthalmic product that contains no antimicrobial preservatives, it is packed in a multi-dose container that maintains product sterility and is as effective in terms of therapy as products available with preservatives.

It is a further object of the present invention to provide a pharmaceutically effective emulsion suitable for ocular application.

In accordance with the above objects of the present invention, an ophthalmic, preservative-free pharmaceutical emulsion is provided comprising Cyclosporine as active ingredient, a tonicity agent, an emulsifying agent, a viscosity modifying agent, an oily component and one or more pH adjusting agents.

A preferred object of the present invention is to provide a simpler and cost effective process for preparing a stable and sterilized Cyclosporine ophthalmic emulsion.

According to another embodiment of the present invention, a process for the preparation of a preservative-free ophthalmic emulsion containing Cyclosporine is provided and it comprises the following steps:
Preparation of primary emulsion by mixing an oil phase comprising castor oil and Cyclosporine with a water phase comprising polysorbate 80 using high shear or high pressure homogenizer and sterilization by filtration.
Preparation of carbomer copolymer type A phase and sterilization by heat.
Final mixing of primary emulsion and carbomer phase of previous steps using magnetic stirrer or high shear homogenizer.

Other objects and advantages of the present invention will become apparent to those skilled in the art in view of the following detailed description.

### DETAILED DESCRIPTION OF THE INVENTION

For the purpose of the present invention, a pharmaceutical composition comprising an active agent or a combination of active agents is considered “stable” if said agent or combination of agents degrades less or more slowly than it does on its own or in known pharmaceutical compositions.

Ocular administration of drugs is primarily associated with the need to treat ophthalmic diseases. Eye is the most easily accessible site for topical administration of a medication. Ophthalmic preparations are sterile products essentially free from foreign particles, suitably compounded and packaged for instillation into the eye. They are easily administered by the nurse or the patient himself, they have quick absorption and effect, less visual and systemic side effects, increased shelf life and better patient compliance.

Antimicrobial preservatives are added to aqueous preparations that are required to be sterile, such as in ophthalmic solutions. The use of preservatives in topical ophthalmic treatments is ubiquitous for any product that is to be used more than once by the patient as they prevent any microbes that may enter into the product after its first use from allowing those microbes to grow and infect the patient on a later use of the product. Although providing effective biocidal properties with well tolerated short-term use at low concentrations, preservatives can cause serious inflammatory effects on the eye with long-term use in chronic conditions, such as glaucoma or potentially ocular allergies.

Antimicrobial preservatives are not found in single use vials of ophthalmic solutions since they are manufactured aseptically or are sterilized and the products are used once and the dispenser is thrown away.

Preservative-free single dose containers most often are presented as blow-fill-seal (BFS) containers. The user takes the plastic vial and tears or cuts the plastic tip, inverts the vial and squeezes the ophthalmic liquid into the eye. Disadvantages of these systems are linked to the quite complicated filling technology, the need to overfill and amount of material needed for each dose. With an average drop size of -35 µl and the standard commercial volume of 400-500 µl, five times the required drug quantity ends up being discarded in case of single dose containers. Additionally, a big amount of packaging material is required associated with high manufacturing costs. A further disadvantage is that, despite numerous technical improvements were made by some manufacturers, the edges around the tip of the opened dropper of disposable, single-dose container are still very sharp, which may cause an accident to the patients eye.

As the use of preservative containing eye drops has been implicated in the development or worsening of ocular surface disease, there is a tendency to limit their use by reducing their concentration as much as possible in eye drops.

Today a range of technical solutions are available to overcome this issue and provide bacterial protection mechanisms. The highest risk of contamination obviously comes from the tip from which the solution exits the container, because it may come in contact with skin and mucosa as well as with infected body fluids. Solutions to prevent contamination via the tip divide into two distinct groups:

1. Containers having “oligodynamic effect” have an open tip release metal ions into the formulation that are toxic to bacteria. Examples include the use of silver wire in the tip of the actuator, a silver coated spring and ball. These components release silver ions into the formulation, which is a time dependent process. The system is able to keep microorganisms down between long dosing intervals, even when the tip is immersed into bacterial contaminated fluid. Silver ions are widely used for their antiseptic properties and even when used for wound dressings, it is safe and no adverse effects are attributed to this treatment. One general limitation of course must be considered: the silver ions may react with certain ions in the formulation and may form precipitates - such as with chloride ions.

2. Containers that use a “mechanical effect” to prevent contamination. Typically this is called “tip seal technology” and is a simple spring loaded valve located directly below the opening of the tip orifice that does not allow any microbes to migrate from any surfaces or contacted liquids into the system; the orifice is sealed under resting conditions. The tip seal keeps the system closed until a defined pressure is reached then the system will open and the formulation is forced through the orifice with a higher pressure than needed to open the valve. When the pressure drops at the end of the actuation the tip seal will immediately close the orifice with an outward movement. So no backflow of potentially contaminated medication or other liquid is possible.

Additionally to protect the integrity of the solution such devices may also have a system to prevent bacteria entering when the system vents. So after use a negative pressure develops inside the container and air may flow back into the container which may carry air bom bacteria. Integrity is achieved by a “mechanical effect” and may be one or more of the following:

1. Collapsible internal bag to contain the solution. The use of an internal collapsible bag to contain the systems avoids any negative pressure developing.

2. Filters, these simply filter the air and trap any air bom bacteria.

3. Unvented containers- these are containers that do not allow any air to come back into the container at all. Negative pressure continues to build throughout the use of the product without affecting the performance of the container to deliver the solution

The present invention provides ophthalmic formulations that are completely free of preservatives. Such formulations are packed in containers that enable to deliver preservative-free formulations while providing shelf life similar to traditional formulations. The containers of the present invention ensure that medication is kept germ-free even after multiple uses.

Patient compliance is greatly increased as the pumps of the present invention permit them to use preservative-free eye drops without worrying about the potential side effects caused by some preservatives and the related short- and long-term consequences, such as pain or discomfort, foreign body sensation, stinging or burning, dry eye sensation, ocular surface breakdown.

We have found that the design of the tip of the container produce a highly accurate drop size with low variability of drop volume between each drop dispensed.

Therefore, we present as a feature of the present invention a multi-use ophthalmic product comprising a container with an integral bacterial protection system and which has a dispensing tip, wherein the ratio of the inner to the outer diameter of the dispensing tip is from 1:1 to 1:6, and the container having an ophthalmic composition that is dispensed from the tip into the eye of a patient wherein the ophthalmic composition is a preservative-free aqueous solution and contains pharmaceutically acceptable excipients.

Studies of of drop size, assay absorption and actuation force indicated that preservative free packaging of the present invention is suitable to be used with Cyclosporine ophthalmic emulsion.

Emulsion may be defined as a biphasic system consisting of two immiscible liquids usually water and oil, one of which is finely subdivided and uniformly dispersed as droplets throughout the other. Since an emulsion is a thermodynamic system, a suitable emulsifying agent is required to stabilize it. It has two phases: i) oil phase and ii) water phase. In other way i) external phase and ii) internal phase. The phase which makes globules or droplets is known as internal phase or disperse phase and other is external or continuous phase. Oil can be present as internal and external phase and water also as internal or external phase. Emulsion is normally opaque. Particle sizes of emulsion are from 0.1 to 100 µm.

Emulsification is the process by which the dispersed phase is broken up into small droplets. Normally a coarse premix is created by rapid mixing of the ingredients. This is sufficient to break up the dispersed phase into large droplets, and allow adsorption of the emulsifiers prior to final emulsification. According to the present invention two main methods/principles are particularly preferred to homogenize the emulsion. A mechanical method under high shear to break up droplets and high pressure homogenizer that forces the premix through a narrow orifice or valve at high pressures (typically 10-100 MPa). Forcing the emulsion through a valve at high pressure creates turbulence and very high shear forces, thus breaking up the droplets.

Extensive studies have been conducted in order to develop a process that provides a stable and sterilized Cyclosporine emulsion for ophthalmic use that is also economic and feasible for commercial scale preparation. A three-step process was developed comprising the preparation of an emulsion by mixing an oil phase comprising Cyclosporine with a water phase using high shear or high-pressure homogenizer. The preparation of a separate aqueous phase comprising a viscosity enhancing agent and the final mixing of the emulsion and viscosity enhancing agent phase using magnetic stirrer or high shear homogenizer.

The ophthalmic, preservative-free emulsion of the present invention comprises Cyclosporine as active ingredient and one or more other components in amounts adequate to facilitate the effectiveness of the compositions. Examples of such other components include tonicity agents, emulsifying agents, emulsion stabilizing agents, viscosity modifying agents, oily materials that solubilize Cyclosporine, acid and/or bases to adjust the pH of the compositions.

The oily component may be considered the discontinuous phase in the Cyclosporine emulsions of the present invention with the water or aqueous phase being considered the continuous phase in such emulsions. Examples of useful oily materials include olive oil, arachis oil, castor oil, mineral oil. The present invention preferably comprises castor oil in an amount of 1%-2% (w/v), most preferably 1.25% w/v.

In general emulsifier components include a hydrophobic and a hydrophilic component. The emulsifier component must be present in an amount effective in forming the present emulsion and maintaining the oily component in emulsion with aqueous component. Suitable emulsifier components include polysorbate 80, polyalkylene oxide ethers of alkyl alcohols and alkylphenols. The present invention preferably comprises polysorbate 80 in an amount of0.25%-2% (w/v), most preferably 0.50%-1% w/v.

Useful tonicity agents in the present invention include mannitol, glycerine, sorbitol. The present invention preferably comprises glycerine in an amount of 1.5%-2.5% (w/v), most preferably 2.20 % w/v.

The compositions of the present invention include viscosity modifying agents such as cellulose polymers, carbomers, alginates, xanthan gums. Such viscosity modifying agents are employed in an amount effective to provide a desired viscosity to the present compositions. The present invention preferably comprises carbomer copolymer type A that also promotes the stabilization of the emulsion. Carbomer is present in an amount of 0.03%-1% (w/v), most preferably 0.05 % w/v.

The pH of the emulsions can be adjusted using sodium hydroxide and/or hydrochloric acid to a physiological pH level. The pH of the emulsions of the present invention is in the range of about 6 to about 10, preferably about 6.5 to about 8 and more preferably from 6.8 to 7.6. The pH can be adjusted in the carbomer phase or in the final product or both.

Pharmaceutical products intended for ophthalmic use must be sterile. Sterilization refers to any process that eliminates, removes, kills, or deactivates all forms of life and other biological agents (such as fungi, bacteria, viruses, spore forms, prions, unicellular eukaryotic organisms such as Plasmodium, etc.) present in a specified region, such as a surface, a volume of fluid, or medication.

Sterilization can be achieved through various means including: heat, chemicals, irradiation, high pressure, and filtration. Sterilization is distinct from disinfection, sanitization, and pasteurization, in that sterilization kills, deactivates, or eliminates all forms of life and other biological agents which are present.

Water at high pressure level is used in moist heat sterilization. Autoclave is the instrument in which this process is carried out. The temperature of the steam in this method is lower when compared with dry heat sterilization, but the high pressure helps with effective sterilization to take place.

Through moist heat sterilization, the most resistant of the spores require a temperature of 121° C. for around half an hour. It is a more effective method when compared with dry heat sterilization. This can be supported by the fact that through moist heat, sterilization can be achieved at lower temperatures in a shorter duration.

In dry heat sterilization, dry heat is used for sterilizing different materials. Heated air or fire is used in this process. As compared to the moist heat sterilization, the temperature in this method is higher. The temperature is usually higher than 356° F. or 180° C. Dry heat helps kill the organisms using the destructive oxidation method. This helps destroy large contaminating bio-molecules such as proteins. The essential cell constituents are destroyed and the organism dies. The temperature is maintained for almost an hour to kill the most difficult of the resistant spores.

Fluids that would be damaged by heat, irradiation or chemical sterilization, such as drug products, can be sterilized by microfiltration using membrane filters. This method is commonly used for heat labile pharmaceuticals. Membrane filters used in production processes are commonly made from materials such as mixed cellulose ester or polyethersulfone (PES). The filtration equipment and the filters themselves may be purchased as pre-sterilized disposable units in sealed packaging or must be sterilized by the user, generally by autoclaving at a temperature that does not damage the fragile filter membranes. To ensure proper functioning of the filter, the membrane filters are integrity tested post-use and sometimes before use. The nondestructive integrity test assures the filter is undamaged and is a regulatory requirement. Typically, terminal pharmaceutical sterile filtration is performed inside of a cleanroom to prevent contamination.

As emulsions are inherently thermodynamically unstable it is very difficult to produce a stable pharmaceutical emulsion that confers adequate treatment to the patient.

The process by which an emulsion completely breaks (coalescence), i.e., the system separates into bulk oil and water phases, is generally considered to be governed by four different droplet loss mechanisms, i.e., Brownian flocculation, creaming, sedimentation flocculation and disproportionation. The first three are the primary methods by which emulsions are destabilized but all four processes may occur simultaneously and in any order.

The present invention has successfully overcome such processing difficulties by applying a step-by-step sterilization in order to obtain a stable and sterile emulsion. More specifically, a primary emulsion was sterilized by filtration and was subsequently mixed with viscosity modifying agent sterilized by heat. The final mixing resulted in a stable emulsion with desired characteristics.

The target of homogenization process is to obtain a primary emulsion with Z-Av of 90-160 nm measured at 1:10 dilution.

The Z-Average size or Z-Average mean used in dynamic light scattering is a parameter also known as the cumulants mean. It is the primary and most stable parameter produced by the technique. The Z-Average mean is the best value to report when used in a quality control setting as it is defined in ISO 13321 and more recently ISO 22412 which defines this mean as the ‘harmonic intensity averaged particle diameter’. The Z-average size will only be comparable with the size measured by other techniques if the sample is monomodal (i.e. only one peak), spherical or near-spherical in shape, monodisperse (i.e. very narrow width of distribution), and the sample is prepared in a suitable dispersant, as the Z-Average mean size can be sensitive to even small changes in the sample, e.g. the presence of a small proportion of aggregates. It should be noted that the Z-average is a hydrodynamic parameter and is therefore only applicable to particles in dispersion or molecules in solution.

### EXAMPLES

Example 1: A preferred preservative-free ophthalmic composition comprising Cyclosporine according to the present invention (Composition 1) is illustrated in Table 1 below:

TABLE 1

Composition 1

| | |
|---|---|
| Ingredients | % w/v |
| Cyclosporine | 0.05 |
| Glycerine | 2.20 |
| Castor oil | 1.25 |
| Polysorbate 80 | 1.00 |
| Carbomer copolymer type A | 0.05 |
| Sodium hydroxide | q.s to adjust pH |
| Purified water | q.s to 100% |

The manufacturing process as followed for the preparation of Composition 1 consists of the following steps:
Preparation of oil phase by mixing Cyclosporine, castor oil and glycerine.
Preparation of aqueous phase by dissolving polysorbate 80 in water.
Preparation of primary emulsion by mixing oil phase with aqueous phase using homogenizer to achieve desired characteristics.
Sterilization of primary emulsion by filtration process (PES membrane).
Preparation of carbomer phase by dissolving carbomer copolymer type A in water.
Heat sterilization of carbomer phase.
Final mixing of carbomer phase and primary emulsion phase using homogenizer to achieve desired final product characteristics.

While the present invention has been described with respect to the particular embodiment, it will be apparent to those skilled in the art that various changes and modifications may be made in the invention without departing from the spirit and scope thereof, as defined in the appended claims.

1. A preservative-free ophthalmic pharmaceutical emulsion comprising a therapeutically effective quantity of Cyclosporine.

2. The preservative free ophthalmic pharmaceutical emulsion according to claim 1, wherein the quantity of Cyclosporine is about 0.05% by weight.

3. The preservative free ophthalmic pharmaceutical emulsion according to claim 1 further comprising adequate amounts of a tonicity agent, on oily material, an emulsifying agent, an emulsion stabilizing agent and acid or bases for pH adjustment.

4. The preservative-free ophthalmic pharmaceutical emulsion according to claim 3, wherein the tonicity agent is glycerine, the oily material is castor oil, the emulsifying agent is polysorbate 80, the emulsion stabilizing agent is carbomer copolymer type A and the pH adjuster is sodium hydroxide.

5. The preservative-free ophthalmic pharmaceutical emulsion according to claim 4, wherein the amount of castor oil is from 1% to 2% w/v, the amount of polysorbate 80 is from 0.25% to 2%, the amount of glycerine is from 1.5% to 2.5% and the amount of carbomer copolymer type A is from 0.03% to 1%.

6. The preservative-free ophthalmic pharmaceutical emulsion according to claim 1, wherein the pH is in range of 6.5 to about 8.

7. The preservative-free ophthalmic pharmaceutical emulsion according to claim 1 in the form of eye drops.

8. The preservative-free ophthalmic pharmaceutical emulsion according to claim 1, wherein it is packed in a multi-use container equipped with an integral bacterial protection system.
